(19) United States
(12) Patent Application Publication
(10) Pub. No.: US 2022/0031379 A1
(43) Pub. Date: Feb. 3, 2022

(54) SINGLE INSTRUMENT ELECTROSURGERY APPARATUS AND ITS METHOD OF USE

(71) Applicant: MEDTRONIC INC., Minneapolis, MN (US)

(72) Inventors: REBECCA SEIDEL, Hudson, WI (US); Jorie Soskin, Edina, MN (US); Sean Chen, Plymouth, MN (US); Satish Pulapura, Bridgewater, NJ (US); Christian S. Nielsen, River Falls, WI (US)

(73) Assignee: MEDTRONIC INC,, Minneapolis, MN (US)

(21) Appl. No.: 17/346,479

(22) Filed: Jun. 14, 2021

Related U.S. Application Data
(60) Provisional application No. 63/058,922, filed on Jul. 30, 2020.

(51) Int. Cl. A61B 18/14; A61L 2/10

(52) U.S. Cl. CPC A61B 18/14; A61B 2018/00601; A61B 2018/1412; A61L 2/10

(57) ABSTRACT

A surgical instrument includes a body extending along a longitudinal axis between opposite proximal and distal end surfaces. The distal end surface includes a first cavity and a second cavity. The first cavity includes a first light bulb disposed therein. The second cavity includes a second light bulb disposed therein. A shaft includes opposite proximal and distal ends. The proximal end is coupled to the body. A blade is coupled to the distal end. The first light bulb is an ultraviolet (UV) light bulb and the second light bulb is configured to emit visible light. Systems and methods of use are disclosed.

### TECHNICAL FIELD

The present disclosure generally relates to electrosurgery generally and more specifically to an electro surgical instrument.

### BACKGROUND

Electrosurgery is a well-known technology utilizing an applied electric current to cut, ablate or coagulate human or animal tissue. See U.S. Pat. No. 7,789,879 issued to Daniel V. Palanker et al., incorporated herein in its entirety by reference. Typical electro surgical devices apply an electrical 15 potential difference or a voltage difference between a cutting electrode and a portion of the patient's grounded body in a monopolar arrangement or between a cutting electrode and a return electrode in bipolar arrangement, to deliver electrical energy to the operative field where tissue is to be treated. The voltage is applied as a continuous train of high frequency pulses, typically in the RF (radio frequency) range.

The operating conditions of electro surgical devices vary, see the above-referenced patent, in particular a configuration of the cutting electrode is described there whereby a conductive liquid medium surrounding the electrode is heated by the applied electric current to produce a vapor cavity around the cutting portion of the electrode and to ionize a gas inside a vapor cavity to produce a plasma. The presence of the plasma maintains electrical conductivity between the electrodes. The 30 voltage applied between the electrodes is modulated in pulses having a modulation format selected to minimize the size of the vapor cavity, the rate of formation of vapor cavity and heat diffusion into the material as the material is cut with an edge of the cutting portion of the cutting electrode.

The operating principle thereby is based on formation of a thin layer of a plasma along the cutting portion of the cutting electrode. Typically, some sort of conductive medium, such as saline solution or normally present bodily fluids, surround the cutting portion of the electrode such that the liquid medium is 40 heated to produce a vapor cavity around the cutting portion. During heating an amount of the medium is vaporized to produce a gas inside a vapor cavity. Since typically the medium is saline solution or bodily fluids, the gas is composed primarily of water vapor. The layer of gas is ionized in 45 the strong electric field or on the cutting electrode to make up the thin layer of plasma. Because the plasma is electrically conductive, it maintains electrical conductivity.

The energizing electrical energy modulation format in that patent includes pulses having a pulse duration in the range of 50 10 microseconds to 10 milliseconds. Preferably the pulses are composed of minipulses having a minipulse duration in the range of 0.1 to 10 microseconds and an interval ranging from 0.1 to 10 microseconds between the minipulses. Preferably the minipulse duration is selected in the range substantially 55 between 0.2 and 5 microseconds and the interval between them is shorter than a lifetime of the vapor cavity. The peak power of the minipulses can be varied from minipulse to minipulse. Alternately, the minipulses are made up of micropulses where each micropulse has a duration of 0.1 to 1 60 microsecond.

Preferably the minipulses have alternating polarity, that is exhibit alternating positive and negative polarities. This modulation format limits the amount of charge transferred to the tissue and avoids various adverse tissue reactions such as 65 muscle contractions and electroporation. Additional devices for preventing charge transfer to the biological tissue can be employed in combination with this modulation format or separately when the method is applied in performing electro-surgery. This pulsing regime is not limiting.

Typically, the temperature of the cutting portion of the electrode is maintained between 40 and 1,000° C.

That patent also describes particular shapes of the electrode and especially its cutting portion in terms of shape and dimensionality. Such electro surgical devices provide several surgical techniques, including cutting, bleeding control (coagulation) and tissue ablation. Typically, different types of electrodes and energizing formats are used for various purposes since the amount of energy applied and the type of tissue being worked on differ depending on the surgical technique being used.

Further, it is known in the field for a single electrosurgery hand piece to have detachable electrodes, such as for instance a cutting electrode and a coagulation electrode. At any one time, only a single electrode is attached to the hand piece, see U.S. Pat. No. 5,984,918 issued to Garito et al. where multiple sized electro surgical electrodes are connected to a handle using a collet member.

Therefore, a known technical problem is that during a surgical procedure, the surgeon must switch between various types of electrosurgical equipment, at least by changing the electrode type. This is typically done by swapping between various electrodes either by changing the electrode portion applied to the body as in Garito et al., or by using entirely different sets of equipment for cutting and coagulation or ablation.

It has been found by the present inventors that this is undesirable and a better system would provide several types of surgical techniques using a single electrosurgical apparatus.

### SUMMARY

An apparatus for electrosurgery in accordance with the invention has been found to reduce operating time, increase ease of use of the equipment, and combine several surgical techniques in one device, including especially cutting and coagulation. In surgery, typically cutting of tissue in the operative field is followed by coagulation of the remaining tissue in the resulting wound to prevent bleeding. Coagulation generally refers to heating the tissue surface so as to seal off small severed blood vessels that would otherwise leak blood into the wound. Coagulation is necessary to prevent blood loss and also because blood leaking into the wound obscures the surgeon's view of the operative field.

The present electrosurgical apparatus provides what is referred to as single instrument surgery and carries out both precision resection (cutting) as well as enhanced coagulation (bleeding control). A typical use is in transcolation, for joint replacement surgery. In some embodiments, this apparatus includes an integrated feature to suck out blood, fluids, smoke, etc. from the operative field to keep the operative field clear, or to supply fluid such as saline solution to the operative field.

In one embodiment the present apparatus includes a hand unit which is mostly conventional for grasping by the surgeon, and which is conventionally coupled at its proximal portion by an electric cable to a control unit which provides the energizing electric pulses or current. The hand unit includes controls including at least one switch or button. The hand unit terminates at its distal portion in a conventional electrosurgical blade (electrode) which is intended for a first electrosurgical procedure such as the cutting (dissection) of tissue, thereby forming the primary assembly. In one embodiment, this electrode is a conventionally shaped electrosurgical blade intended for cutting soft tissue and is typically of metal most of the surface area of which is electrically insulated such as by a thin layer of glass.

The type of electrical energy applied to this blade by the control unit is, e.g., as described in the above-referenced patent so as to provide plasma type conditions at the electrode tip for tissue cutting, but this is not limiting. In one embodiment, this blade has a 3.0 mm wide spatula shaped tip mounted on a variable length (extendable) shaft. An example is in the PEAK PlasmaBlade® 3.05 surgical instrument supplied by PEAK Surgical, Inc., of Palo Alto, Calif. which has a telescoping electrode shaft and a spatula shaped electrode tip which is 3 mm wide, and an integrated aspiration feature. This device includes the hand unit.

In one embodiment, the present apparatus is a monopolar type cutting device (like the PEAK PlasmaBlade instrument) whereby the return current path is via a grounding pad or other return electrode affixed to the patient's body remote from the electrosurgical instrument. In other embodiments, the present apparatus is a bipolar type where the return electrode is located on or near the main electrode and is an integral part of the electrosurgical apparatus, as also well known in the field.

The secondary assembly of the present apparatus, in one embodiment, is intended for a second electrosurgical procedure such as tissue coagulation. It terminates at its distal portion in its own electrode blade or tip which in one case is hemispherical (ball shaped) and which is the distal end of an electrode shaft which is at least partially insulated. The proximate portion of the electrode shaft terminates in a housing which fits closely around the electrode shaft and provides heat and electrical insulation and a finger grip region. However, the housing itself is not intended to be held by the surgeon when the apparatus is in use. Instead, this housing fits snugly over the electrode blade of the primary assembly so that the electrode of the primary assembly also makes electrical contact with the electrode shaft of the secondary assembly. The electrical energy (pulsing) or continuous wave regime applied to the electrode of the secondary assembly (via the hand unit) may differ from that supplied to the primary assembly. The selection of the electrical energy regime is conventionally performed by the surgeon by manipulating controls on the control unit or on the hand unit.

For coagulation, the duty cycle regime of the applied electrical energy is, e.g., in the range of 12% to 19%. With associated peak to peak voltages in the range of 1300 to 5000 volts, the coagulation effect can be achieved using the electrode of the secondary assembly with conventional settings of the associated pulse generator of “cut”, “coagulation” or “blend.” Since the surface area of this coagulation electrode is large with respect to the applied voltage, the effect is resistive heating of the tissue rather than plasma generation that would ablate (cut) the tissue. For coagulation, generally the electrode is heated to about 100° C. so as to heat fluids in the tissue so the tissue in contact with the active portion of the electrode desiccates or stops bleeding:

When the secondary assembly is thus mounted to the primary assembly, the apparatus is suitable for coagulation since the primary assembly's electrode blade is now hidden and only serves as a mechanical mounting and electrical connection to the electrode of the coagulation (secondary) assembly. The secondary assembly fits over the distal portion of the primary assembly, e.g., with a snap (friction) fit so the secondary assembly can be readily attached and removed by the surgeon during surgery, without unscrewing or any tool. Thereby the surgeon can quickly switch between cutting and coagulation procedures, with essentially the same apparatus. When the surgeon mounts the coagulation (secondary) assembly to the primary assembly, he also may reset the control unit to supply electrical energy (pulsing or continuous) in the desired modulation regime suitable for tissue coagulation by the coagulation electrode.

In some embodiments, the two electrodes each carry a non-stick coating on their exposed (non insulated) portions. The coagulation electrode may be a ball, tube, screen, suction coagulator, or forceps type electrode. Also, the secondary (coagulation) assembly may be provided with a drip chamber near its distal portion or a perforated shaft so as to deliver fluid to the operative field, such as saline, or to provide aspiration. In some embodiments, a conventional channel or other type of passage such as a tube is provided for aspiration of smoke and/or fluid from the wound or fluid delivery. This passage (or passages) may be provided in only the secondary or primary assembly or in both in a fluid communication fashion. In some embodiments, the secondary assembly near its distal portion defines one or more aspiration ports, such as three such ports arranged around the circumference of the assembly and spaced at 120 degrees from one another, all in communication with the aspiration channel.

In one embodiment, the coagulation assembly's electrode shaft is bendable so that the surgeon can bend it and it remains in the bent position for ease of reaching portions of the operative field. The housing of the secondary assembly defines exterior finger grip ridges (ribs) in one embodiment so as to make it easier for the surgeon to attach and detach it from the primary assembly.

Therefore the secondary assembly, which in one embodiment is intended for coagulation, attaches to the primary assembly and by making electrical and mechanical contact thereto, conducts the electrical energy originating at the control unit, via the primary assembly electrode, to the tip of the coagulation assembly electrode. The shape of the electrode of the primary assembly is not limited to being a blade, but may take any other typical shape, such as a ball, tube, screen, suction coagulator, or forceps. In one embodiment, the mechanical and electrical contact between the two assemblies is maintained at least partly by a spring in the housing of the secondary assembly.

Further, in other embodiments the functionality of the primary and secondary assemblies is reversed, so the primary electrode is for coagulation and the secondary electrode is for cutting. In other embodiments, the two electrodes have other intended uses in terms of electrosurgical procedures.

Advantages of the present device include reduced cost in use since there is no need to supply saline solution to the operative field. This also reduces smoke production, making the surgery easier. Further there is no need for a separate aspiration device since aspiration is integrated into the device.

In one embodiment, in accordance with the principles of the present disclosure, a surgical instrument comprises a body extending along a longitudinal axis between opposite proximal and distal end surfaces. The distal end surface comprises a first cavity and a second cavity. The first cavity comprises a first light bulb disposed therein. The second cavity comprises a second light bulb disposed therein. A shaft comprises opposite proximal and distal ends. The proximal end is coupled to the body. A blade is coupled to the distal end. The first light bulb is an ultraviolet (UV) light bulb and the second light bulb is configured to emit visible light.

In one embodiment, in accordance with the principles of the present disclosure, a surgical method comprises: providing a first surgical instrument including a body extending along a longitudinal axis between opposite proximal and distal end surfaces, the distal end surface comprising a first cavity and a second cavity, the first cavity comprising a first light bulb disposed therein, the second cavity comprising a second light bulb disposed therein, the primary assembly comprising a shaft, the shaft comprising opposite proximal and distal ends, the proximal end being coupled to the body, the primary assembly comprising a blade coupled to the distal end, wherein the first light bulb is an ultraviolet (UV) light bulb and the second light bulb is configured to emit visible light; sterilizing the skin and tissue using the first light bulb; illuminating the sterilized tissue using the second light bulb; and cutting the sterilized and illuminated tissue with the blade.

In one embodiment, in accordance with the principles of the present disclosure, a surgical instrument comprises a body extending along a longitudinal axis between opposite proximal and distal end surfaces. The distal end surface comprises a plurality of spaced apart first cavities and a plurality of spaced apart second cavities. The first and second cavities are each disposed radially about the longitudinal axis such that each of the first cavities is positioned between two of the second cavities and each of the second cavities is positioned between two of the first cavities. The first cavities each comprise a first light bulb disposed therein. The second cavities each comprise a second light bulb disposed therein. A shaft comprising opposite proximal and distal ends. The proximal end is coupled to the body. An electrode is coupled to the distal end. An insulating portion is positioned between the shaft and the electrode. The first light bulbs are ultraviolet light bulbs and the second light bulbs are light emitting diodes.

### BRIEF DESCRIPTION OF THE DRAWINGS

The present disclosure will become more readily apparent from the specific description accompanied by the following drawings, in which:

FIG. 1 shows an embodiment of the present apparatus as fully assembled, including the coagulation (secondary) assembly and the hand piece and electrode of the primary (main or cutting) assembly;

FIG. 2 shows the primary assembly separated from the coagulation assembly of FIG. 1;

FIG. 3 shows the distal end of the primary assembly, including its cutting electrode;

FIG. 4 shows an exploded view of the coagulation assembly;

FIG. 5 shows detail of the coagulation assembly in a cross-sectional view;

FIG. 6 shows detail of the tip of the coagulation assembly'

FIG. 7 shows an “X-ray” view of the distal end of the primary assembly and the proximal end of the coagulation assembly; and

FIG. 8 shows a perspective view of one embodiment of the primary assembly, in accordance with the principles of the present disclosure.

Like reference numerals indicate similar parts throughout the figures.

### DETAILED DESCRIPTION

The exemplary embodiments of the apparatus disclosed are discussed in terms of medical devices for the general procedure particularly operating in a surgical cavity is needed, where potential infection complication rates is high. In some embodiment, the medical device is used to create a surgical pocket for implanting an electronic implantable devices such as pacemaker, defibrillator, or neurological stimulators. In some embodiment, the medical device is used in the breast implant surgical procedure. In some embodiment, the surgical device is used for treatment of musculoskeletal disorders and more particularly, in terms of a surgical system and a method for treating a spine. In some embodiments, the systems and methods of the present disclosure comprise medical devices including surgical instruments and implants that are employed with a surgical treatment, as described herein, for example, with a cervical, thoracic, lumbar and/or sacral region of a spine.

In some embodiments, the surgical system of the present disclosure may be employed to treat spinal disorders such as, for example, degenerative disc disease, disc herniation, osteoporosis, spondylolisthesis, stenosis, scoliosis and other curvature abnormalities, kyphosis, tumor, and fractures. In some embodiments, the surgical system of the present disclosure may be employed with other osteal and bone related applications, including those associated with diagnostics and therapeutics. In some embodiments, the disclosed surgical system may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The surgical system of the present disclosure may also be alternatively employed with procedures for treating the lumbar, cervical, thoracic, sacral and pelvic regions of a spinal column. The surgical system of the present disclosure may also be used on animals, bone models and other non-living substrates, such as, for example, in training, testing and demonstration.

The surgical system of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. In some embodiments, as used in the specification and including the appended claims, the term “tissue” includes soft tissue, ligaments, tendons, cartilage and/or bone unless specifically referred to otherwise.

The following discussion includes a description of a surgical system including implants, related components and methods of employing the surgical system in accordance with the principles of the present disclosure. Alternate embodiments are also disclosed. Reference is made in detail to the exemplary embodiments of a surgical system, which are illustrated in the accompanying figures.

The components of the surgical system can be fabricated from biologically acceptable materials suitable for medical applications, including metals, synthetic polymers, ceramics and bone material and/or their composites. For example, the components of the surgical system, individually or collectively, can be fabricated from materials such as stainless steel alloys, aluminum, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL®), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

Various components of the surgical system may have material composites, including the above materials, to achieve various desired characteristics such as strength, rigidity, elasticity, compliance, biomechanical performance, durability and radiolucency or imaging preference. The components of the surgical system, individually or collectively, may also be fabricated from a heterogeneous material such as a combination of two or more of the above-described materials. The components of the surgical system may be monolithically formed, integrally connected, or include fastening elements and/or instruments, as described herein.

The surgical system is employed, for example, with a fully open surgical procedure, a minimally invasive procedure including percutaneous techniques, and mini-open surgical techniques to deliver and introduce instrumentation and/or one or more spinal implants, such as, for example, one or more components of a bone fastener, at a surgical site of a patient, which includes, for example, a spine. In some embodiments, the spinal implant can include one or more components of one or more spinal constructs, such as, for example, interbody devices, interbody cages, bone fasteners, spinal rods, tethers, connectors, plates and/or bone graft, and can be employed with various surgical procedures including surgical treatment of a cervical, thoracic, lumbar and/or sacral region of a spine.

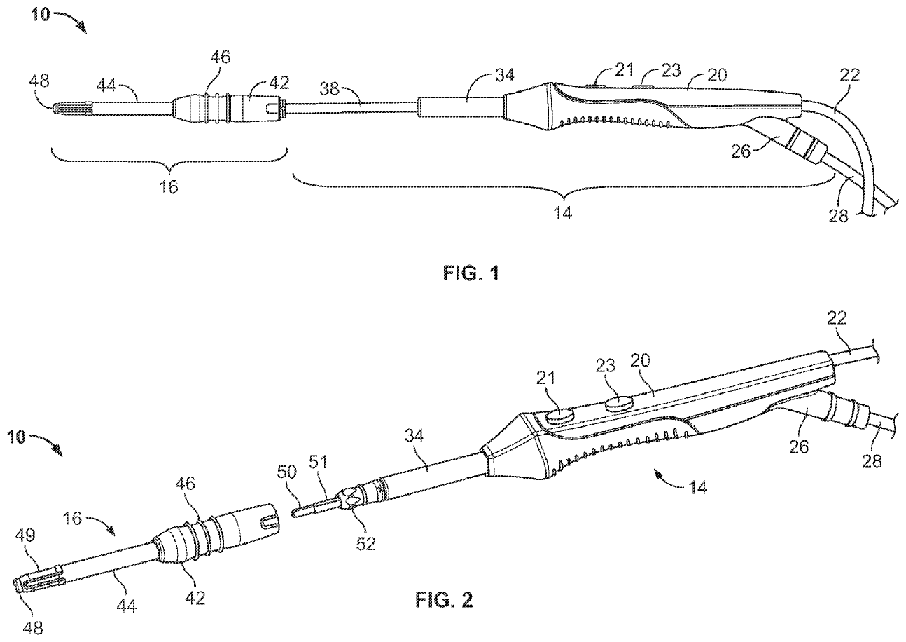

FIG. 1 shows an electrosurgical apparatus 10 in accordance with the invention, having two main portions or assemblies. Primary assembly 14 is intended for tissue cutting and includes a body, such as, for example, a conventional hand piece 20 to which is conventionally coupled an insulated electrical cable 22 for providing the energizing electrical current or pulses. Also provided is extension 26 from which extends conventionally a tube or tubes for aspiration and/or providing fluid. These tubes are connected through suitable channels to a distal portion of the primary assembly. Also shown in FIG. 1 is the boot or seal 34 between the hand piece 20 and shaft 38; boot 34 is typically of electrically insulative material such as silicone, extending from which is the electrode shaft 38. Assembly 14 is, e.g., the PEAK PlasmaBlade device as described above. Conventional cut/coagulation control buttons are respectively at 21, 23.

The second portion of the apparatus 10 is the secondary (here a coagulation “cap”) assembly 16 which includes housing 42 including exterior finger grip ridges 46, and from which extends an insulated shaft 44 terminating in electrode tip 48. Note that dimensions and materials here are largely conventional, as explained hereinafter.

FIG. 2 also shows the two assemblies 14 and 16 in a slightly different view, but detached (demounted) from one another. Generally, the same reference numbers used in different figures here refer to the same or similar structures. In FIG. 2, extension portion 38 of the primary assembly is not visible since it is retracted.

In FIG. 2, since the two assemblies are shown detached, also visible is the base portion 52 of the primary assembly which in one embodiment has its own grip 52 as described and shown further below. Typically grip 52 is, e.g., of a high durometer (hard) polymer material. Blade 50 extends from an insulated portion 51.

In one embodiment, shaft 44 of secondary assembly 16 is bendable. While here assembly 16 has a hemispherical or ball electrode 48, this electrode may have any other shapes, such as tube, screen, or forceps. Moreover, the exposed conductive (non-insulated) portion of electrode 48 may carry a non-stick coating, such as carbon with a protein such as a collagen, or a material such as PTFE or other flouro-polymer. This electrode is metal and glass coated, but the glass defines a large number of voids or micro-cracks which in use define hot spots by increasing the local impedance to the energizing electrical current. So, these hot spots are intended to cause arcing and heating. A typical impedance is 50 to 2K ohms. While this glass insulation wears away as a result of the arcing, this is not problematic due to the use of this electrode for only one surgical procedure. A typical thickness of this glass layer is 0.003 to 0.007 inches (0.076 to 0.178 mm).

In one embodiment, the shaft of the coagulation electrode immediately proximal its tip 48 is surrounded by a drip chamber 49 for supplying fluid to the operative field, supplied via a suitable passage defined through secondary assembly 16 and connecting to a similar passage in the primary assembly 14. This passage and drip chamber provide, for instance, saline solution to the operative field.

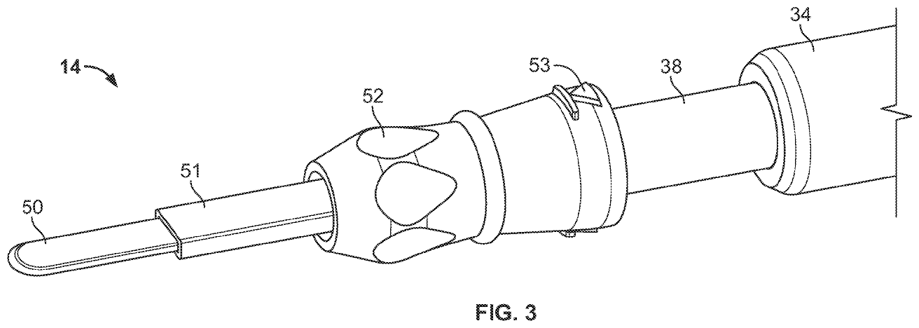

FIG. 3 shows detail of the distal portion of the primary assembly 14, including similar structures as in FIGS. 1 and 2. FIG. 3 also shows in greater detail the grip 52. Arrow indicator 53 is provided so that the operator, such as a surgeon, has a reference indicator that the shaft 38 extends from the boot 34. Attachment of the secondary assembly 16 onto the primary assembly 14 is not orientation specific in this embodiment.

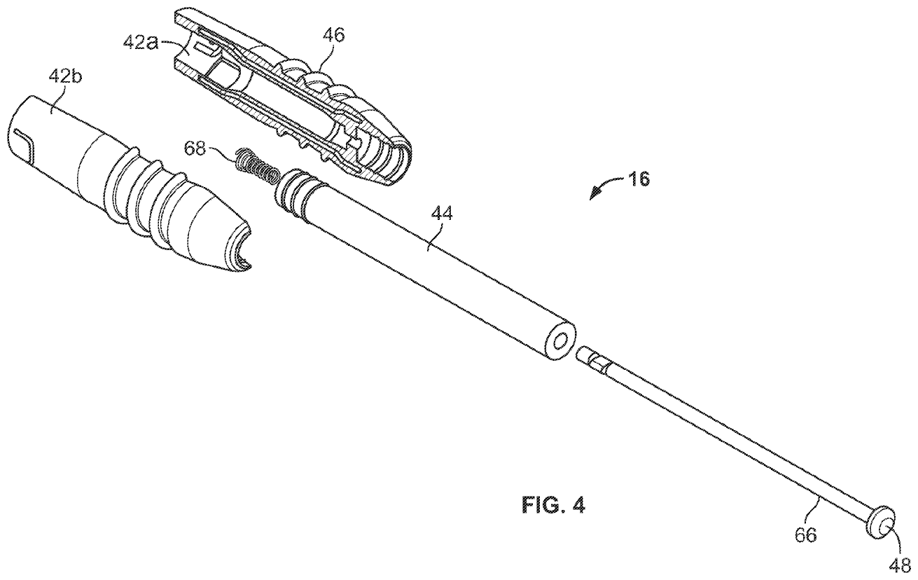

FIG. 4 is an “exploded” view of the coagulation assembly 16. The ball tip 48 of the electrode is the distal portion of a conventional metal (or similar electrically conductive material) shaft 66 which can be bendable. The outer portion 44 of the shaft here is an electrically insulative tubing, such as plastic, which covers most of the length of conductive shaft 66. This tubing 44 may be perforated to deliver saline or serve as an aspiration channel for smoke. In other embodiments it is not so perforated. Spring 68 surrounds and contacts the proximal end of shaft 66, as explained hereinafter. Housing 42 includes two mating portions 42a and 42b, each for instance of plastic.

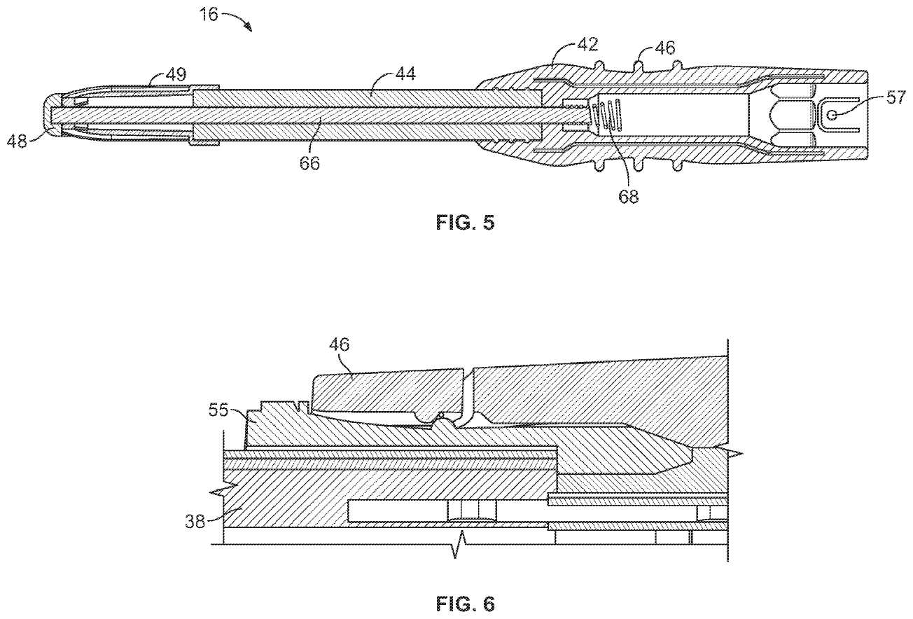

FIG. 5 shows a cross-sectional view of the coagulation assembly 16. FIG. 6 shows detail of the tip of the coagulation assembly 16. As shown, a short distance proximal from ball electrode 48, aspiration port 67 is defined in the shaft 66 and its outer portion 44, for passage of smoke, blood, etc. into an interior channel defined in shaft 66, in this embodiment. There are for instance three such ports disposed around shaft 66, equally spaced apart circumferentially. A typical diameter of the ball electrode is 0.18 inches (4 mm) and the port diameter is typically 0.06 inches (1.5 mm). The outer shaft 44 is electrically and heat insulative, for instance made of plastic, and is typically 0.10 inch (2.5 mm) thick. Some of this insulation extends into the port 67, to prevent debris build up in the port.

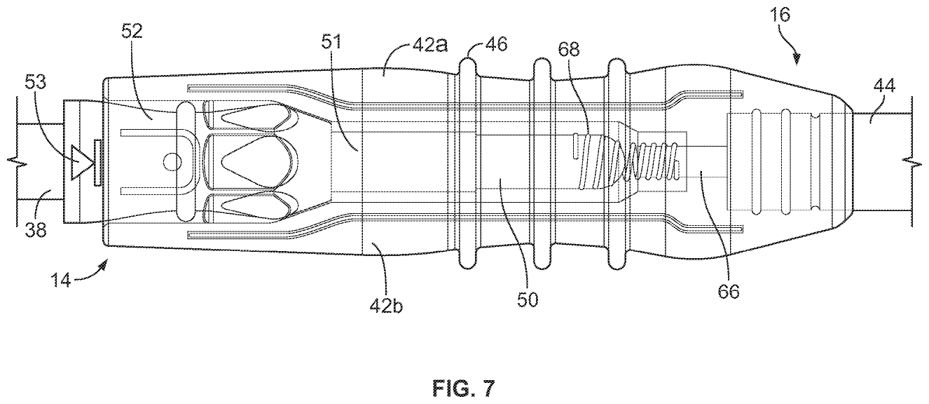

FIG. 7 shows in a “X-ray” view how primary assembly 14 mates with coagulation assembly 16. Again, the reference numbers refer to the same structures as in the other figures. The two housing halves 42a, 42b of the coagulation assembly fit over and engage the grip 52 of the primary assembly 14. The mating is intended to be finger tight so the two assemblies can be attached and detached with normal hand strength. Spring 68 of coagulation assembly 16 fits over and engages blade 50 of the primary assembly 14. Arrow indicator 53 on primary assembly 14 points to an associated indicator mark on the exterior of the coagulation assembly housing 42, as described above. The mating portions of the two assemblies in this embodiment are both rotatably symmetric, so there is no need to align one to the other rotationally.

Other portions of the present electrosurgical system which are conventional are not shown here. Notably the control unit provides the electric current or pulses as explained above and is of the type well known in the field and is electrically coupled via cable 22 to the present apparatus. An example of such a control unit is the PULSAR® Generator power supply supplied by PEAK Surgical, Inc.

Also provided, if needed, is a conventional source of fluid and/or a source of vacuum, for aspiration, as well known in the field. Typically, the electrically non-conductive portions of the apparatus are polymer or plastic in terms of the housings, tubing, etc. and of conventional materials. The insulative tubing is typically heat shrink or silicone material. The two halves 42a, 42b of housing 42 are glued or otherwise fastened together, although in other embodiments, this housing is a single piece of material. As explained above, the coagulation assembly shaft 66 may be of a bendable material, such as a somewhat flexible or annealed metal rod such as, for instance, stainless steel and has a typical diameter of 0.5 to 2 mm.

Typically, the two electrodes are single use (disposable) so as to be used for only a single surgical operation. In particular the entire coagulation subassembly 16 is typically disposable. In terms of the primary assembly 14, the entire assembly is also disposable, or at least its distal portions including the electrode and its shaft are disposable and detachable from the hand piece which then may be reusable.

As described above, the exposed (non-insulated) electrode tips of both the primary assembly and the coagulation assembly in one embodiment carry a non-stick coating. These coatings in one embodiment are conventional polymers or flouro-polymers. In another embodiment they are diamond like carbon which conventionally is one of several forms of an amorphous carbon material formed by deposition.

In other embodiments, the electrode tip coatings are carbon together with a collagen or other protein. For instance, this coating may be carbon graphite with a protein or albumin binder. The thickness of the carbon coating on the metal (or other conductive material) surface of the electrode, as needed to support an electrical discharge, is in the range of 10 μm to 1 mm. Conventional carbon sputtering provides only a thickness of 0.1 μpm, which is inadequate. A pyrolytic carbon deposition method is known from Morrison, Jr. U.S. Pat. No. 4,074,718 incorporated herein by reference in its entirety, forming carbon on an electrode by burning carbohydrate-containing materials deposited on the electrode.

The present coating process is different and first involves providing a mixture of carbon or graphite powder (of any convenient particle size) and a binder. The mixture is 1% to 50% powdered carbon or graphite (by weight or volume), preferably about 30% by volume. The binder is a solution of a protein or similar material such as albumin, gelatin, collagen or other biocompatible material in water or other solvent. For instance, the binder may be a 35% solution by volume of albumin in saline solution.

The bare electrode is briefly dipped into the mixture. The coated electrode is then air dried for, e.g., one minute to one hour at an ambient temperature of 200° C. to 300° C., or until all the solvent has evaporated. Then the coated electrode is placed in an oven for a few seconds to an hour, at a temperature of 200° C. to 600° C. E.g., this baking step takes 5 minutes at 300° C. (Note that the drying and baking can be combined into one step.)

The electrode is then cooled in the air and ready for assembly with the associated components of the apparatus.

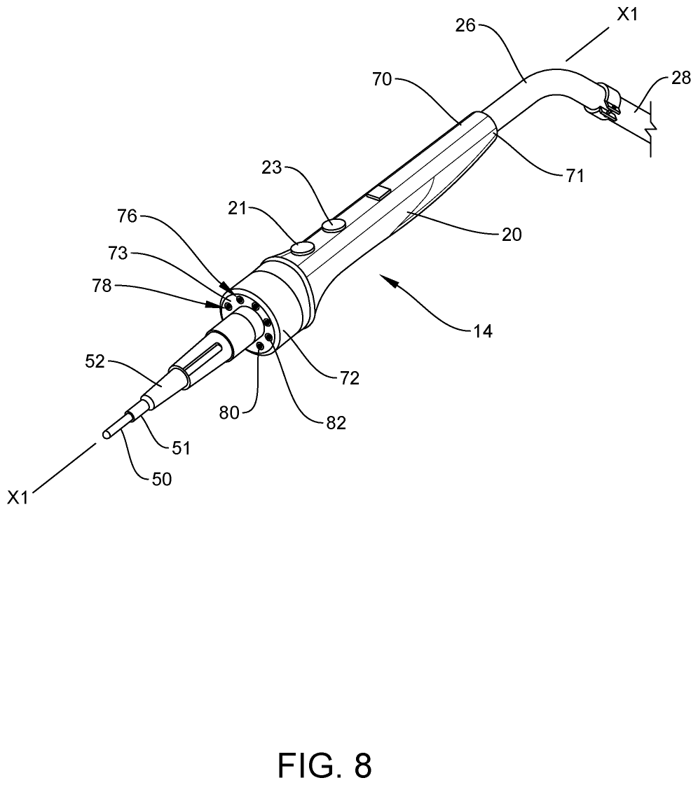

In one embodiment, shown in FIG. 8, apparatus 10 is configured to sterilize tissue that is to be cut by blade 50 and/or tissue adjacent to tissue that is to be cut by blade 50, as discussed herein. In such embodiments, body 20 extending along a longitudinal axis X1 between a proximal end 70 and an opposite distal end 72. End 70 includes an end surface 71 and end 72 includes an end surface 73 that faces away from end surface 71. Extension 26 extends into and/or through end surface 71. In some embodiments, end surface 71 and/or end surface 73 extends perpendicular to axis X1. In some embodiments, end surface 71 and/or end surface 73 may be disposed at alternate orientations, relative to first longitudinal axis X1, such as, for example, transverse, oblique and/or other angular orientations such as acute or obtuse, acute and/or may be offset or staggered.

End surface 72 defines one or a plurality of sockets, such as, for example, first cavities 76 and one or a plurality of sockets, such as, for example, second cavities 78. Cavities 76, 78 each extend into end surface 72 toward end 70. Cavities 76 each include a light source, such as, for example, an ultraviolet (UV) bulb 80 disposed therein. Cavities 78 each include a light source, such as, for example, a bulb 82 disposed therein that is configured to emit visible light. In some embodiments, bulb(s) 82 is/are a light emitting diode (LED). When primary assembly 14 is in use without secondary assembly 16, bulbs 80, 82 provide visible light to illuminate a surgical site and to sterilize tissue at or adjacent to the surgical site. That is, bulb(s) 80 direct UV light to tissue to sterilize such tissue. This allows such tissue to be sterilized prior to performing a surgical procedure, during the surgical procedure and/or after the surgical procedure. Bulb(s) 82 direct visible (white) light to the surgical site to provide better visualization of the surgical site. It is envisioned that bulb(s) 82 can emit the visible light at the same time bulb(s) emit the UV light. It is further envisioned that bulb(s) 82 can emit the visible light before and/or after bulb(s) emit the UV light. In some embodiments, bulbs 80 can be turned on and off using button 21 and bulbs 82 can be turned on and off using button 23 to allow bulbs 82 to be turned on and off independently of bulbs 80, and vice versa. In some embodiments, bulbs 80 and bulbs 82 can be turned on and off simultaneously using button 21 or button 23.

In some embodiments, at least one of bulbs 80 is a UV LED. In some embodiments, at least one of bulbs 80 is an incandescent mercury vapor light bulb. In some embodiments, at least one of bulbs 80 is an incandescent xenon light bulb. In some embodiments, at least one of bulbs 80 is configured to emit UV light in a range of 100 nm to 280 nm (UVC light). In some embodiments, at least one of bulbs 80 is configured to emit UVC light having a wavelength of 254 nm. In some embodiments, at least one of bulbs 82 is a halogen light bulb. In some embodiments, at least one of bulbs 82 is configured to emit white light in a range between about 600 nm to about 400 nm.

In some embodiments, bulbs 80 are positioned in cavities 76 such bulbs 80 are fixed to body 20 and bulbs 82 are positioned in cavities 78 such that bulbs 82 are fixed to body 20. That is, bulbs 80 are positioned in cavities 76 such that bulbs 80 cannot move relative to body 20 without breaking bulbs 80 and//or body and bulbs 82 are positioned in cavities 78 such that bulbs 82 cannot move relative to body 20 without breaking bulbs 82 and/or body 20. In some embodiments, bulbs 80 are positioned in cavities 76 such bulbs 80 are rotatable relative to body 20 and bulbs 82 are positioned in cavities 78 such that bulbs 82 are rotatable to body 20. In some embodiments, bulbs 80 are positioned in cavities 76 such bulbs 80 are fixed to body 20 and bulbs 82 are positioned in cavities 78 such that bulbs 82 are rotatable relative to body 20. In some embodiments, bulbs 80 are positioned in cavities 76 such bulbs 80 are rotatable relative to body 20 and bulbs 82 are positioned in cavities 78 such that bulbs 82 are fixed to body 20.

In some embodiments, bulbs 80 are positioned in cavities 76 such that the UV light emitted by bulbs 80 travels in a direction that is parallel to axis X1 and bulbs 82 are positioned in cavities 78 such that the visible light emitted by bulbs 82 travels in a direction that is parallel to axis X1. In some embodiments, bulbs 80 are positioned in cavities 76 such that the UV light emitted by bulbs 80 travels in a direction that is at an acute or oblique angle relative to axis X1 and bulbs 82 are positioned in cavities 78 such that the visible light emitted by bulbs 82 travels in a direction that is at an acute or oblique angle relative to axis X1. In some embodiments, bulbs 80 are positioned in cavities 76 such that the UV light emitted by bulbs 80 travels in a direction that is parallel to axis X1 and bulbs 82 are positioned in cavities 78 such that the visible light emitted by bulbs 82 travels in a direction that is at an acute or oblique angle relative to axis X1. In some embodiments, bulbs 80 are positioned in cavities 76 such that the UV light emitted by bulbs 80 travels in a direction that is at an acute or oblique angle relative and bulbs 82 are positioned in cavities 78 such that the visible light emitted by bulbs 82 travels in a direction that is parallel to axis X1. In some embodiments, at least one of cavities 76 extends at a different angle relative to axis X1 than at least another one of cavities 76 such that the UV light emitted by at least two of bulbs 80 travels in different directions. In some embodiments, each of cavities 76 extends at a different angle relative to axis X1 such that the UV light emitted by each of bulbs 80 travels in different directions. In some embodiments, at least one of cavities 78 extends at a different angle relative to axis X1 than at least another one of cavities 78 such that the visible light emitted by at least two of bulbs 82 travels in different directions. In some embodiments, each of cavities 78 extends at a different angle relative to axis X1 such that the visible light emitted by each of bulbs 82 travels in different directions.

In some embodiments, the UV light emitted by bulbs 80 has a fixed strength that cannot be adjusted. That is, bulbs 80 either emit no UV light or UV light of a fixed strength. In some embodiments, the strength of UV light emitted by bulbs 80 can be selectively adjusted. In some embodiments, cavities 76 and/or cavities 78 may have various cross section configurations, such as, for example, circular, oval, oblong, triangular, rectangular, square, polygonal, irregular, uniform, non-uniform, variable, tubular and/or tapered. In some embodiments, cavities 76 and/or cavities 78 may have cross section configurations that correspond to that of bulbs 80 and/or bulbs (82). In some embodiments, the engagement of bulbs 80 with cavities 76 and/or the engagement of bulbs 82 with cavities 78 may include threads, mutual grooves, screws, adhesive, nails, barbs, raised elements, spikes, clips, snaps, friction fittings, compressive fittings, expanding rivets, staples, fixation plates, key/keyslot, tongue in groove, dovetail, magnetic connection and/or posts.

It is envisioned that cavities 76 and cavities 78 may be selectively positioned to direct the UV light from bulbs 80 and the visible light from bulbs 82 in a selected manner. In some embodiments, cavities 76 and cavities 78 are each disposed radially about axis X1 such that each of cavities 76 is positioned between two cavities 78 and each of cavities 78 is positioned between two cavities 76. In some embodiments, cavities 76 and cavities 78 are each disposed radially about axis X1 such that only cavities 76 extend about one side or hemisphere of surface 73 and only cavities 78 extend about the other side or hemisphere of surface 73. Other configuration are also contemplated.

In some embodiments, apparatus 10 is free of any shields, such as protective shields between bulbs 80 and patient tissue such that UV light emitted by bulbs 82 can travel directly to patient tissue without passing through any protective shield or other structure. In some embodiments, apparatus 10 includes a shield, such as, for example a filter that is applied to end surface 73 such that UV light emitted by bulbs 82 must travel through the filter before the UV light is able to travel to patient tissue.

In some embodiments, bulbs 80, 82 may be optical fiber with light source at proximate end of tool. In some embodiments, all LED's could be replaced by optical fiber light guides thus providing further design flexibility in the handle. This may include bringing the light source closer to the distal end of the tool, higher light intensity at distal end since there's no limit on light source size/power at proximal end. This approach may include both visible light for increased visibility and wavelengths shorter than 260nm for anti-bacterial function. In some embodiments, light guides could be integral to 26. In some embodiment, the UV stable fibers are fibers available at https://www.lasercomponents.com/de-en/news/transmission-of-uv-light-with-optical-fiber/. In some embodiments, a proximal light source may now be a laser source and would therefore provide very high intensities that allows for short duty cycles (periodic pulses) that limits/manages the exposure of UV radiation to surgeon and supporting staff.

In assembly, operation and use, apparatus 10 that includes the primary assembly 14 shown in FIG. 8 is employed with a surgical procedure for treatment of a spinal disorder affecting a section of a spine of a patient, as discussed herein. That is, apparatus 10 that includes the primary assembly 14 shown in FIG. 8 is employed with a surgical procedure for treatment of a condition or injury of an affected section of the spine, such as, for example, vertebrae.

In use, to treat a selected section of vertebrae, a medical practitioner obtains access to a surgical site in any appropriate manner, such as through incision and retraction of tissues. In some embodiments, apparatus 10 that includes the primary assembly 14 shown in FIG. 8 can be used in any existing surgical method or technique including open surgery, mini-open surgery, minimally invasive surgery and percutaneous surgical implantation, whereby vertebrae are accessed through a mini-incision, or sleeve that provides a protected passageway to the area. Once access to the surgical site is obtained, the particular surgical procedure can be performed for treating the spine disorder.

An incision is made in the body of a patient. In some embodiments, apparatus 10 that includes the primary assembly 14 shown in FIG. 8 is used a cutting instrument to create a surgical pathway for implantation of components of a surgical system at a selected surgical site, such as, for example, one or more vertebrae and/or as a cutting instrument at the surgical site. A preparation instrument can be employed to prepare tissue surfaces of vertebrae as well as for aspiration and irrigation of a surgical region.

Tissue at or adjacent to the surgical site is sterilized using bulbs 80 and/or tissue at or adjacent to the surgical site is illuminated using bulbs 82. In some embodiments, the surgical site is first illuminated using bulbs 82 to enhance visualization to assist a medical practitioner in guiding blade 50 to position blade 50 adjacent to tissue that the medical practitioner intends to cut using blade 50. In some embodiments, bulbs 80 direct UV light to the tissue that intended to be cut before the tissue is in fact cut. In some embodiments, bulbs 80 direct UV light to the tissue that is being cut. That is, bulbs 80 direct UV light to tissue as the tissue is being cut. In some embodiments, bulbs 80 direct UV light to tissue that is adjacent to the cut tissue after the tissue has been cut. In some embodiments, bulbs 82 can direct visible light to the surgical site, including the tissue that is intended to be cut before such tissue is cut, while such tissue is being cut, and after such tissue is cut.

In some embodiments, secondary assembly 16 is coupled to the primary assembly 14 shown in FIG. 8 in the same manner discussed herein for coupling secondary assembly 16 to the primary assembly 14 shown in FIGS. 1-3 and 7. For example, in some embodiments, secondary assembly 16 is coupled to the primary assembly 14 shown in FIG. 8 by mounting housing 42 on blade 50 and shaft 38 such that electrode 48 is coupled to blade 50, as discussed herein with respect to coupling secondary assembly 16 to the primary assembly 14 shown in FIGS. 1-3 and 7. Once secondary assembly 16 is coupled to the primary assembly 14 shown in FIG. 8, a procedure may be performed using electrode 48. In some embodiments, the procedure performed by electrode comprises coagulating tissue adjacent to the tissue that was cut using blade 50. In some embodiments, secondary assembly 16 is removed from the primary assembly 14 shown in FIG. 8 after electrode performs the procedure such that secondary assembly 16 is spaced apart from the primary assembly 14 shown in FIG. 8. In some embodiments, the tissue that is coagulated by electrode 48 can be sterilized using bulbs 80.

Upon completion of a procedure, as described herein, the surgical instruments, assemblies and non-implanted components are removed and the incision(s) are closed. One or more of the components of apparatus 10 can be made of radiolucent materials such as polymers. Radiomarkers may be included for identification under x-ray, fluoroscopy, CT, or other imaging techniques.

In one embodiment, an agent may be disposed, packed, coated, or layered within, on or about the components and/or surfaces of apparatus 10. In some embodiments, the agent may include one or a plurality of therapeutic agents and/or pharmacological agents for release, including sustained release, to treat, for example, pain, inflammation and degeneration.

In some embodiments, the primary assembly 14 shown in FIG. 8 may be configured as a driver, for example. In such embodiments, blade 50 and/or insulated portion 51 can be replaced with a drive bit that is fixed to shaft 38 such that rotation of shaft also rotates the drive bit, wherein the drive bit is configured for disposal in a drive socket of an implant, such as, for example, a bone screw. It is further envisioned that blade 50 and/or insulated portion 51 can be replaced with other tips configured to perform other procedures.

In some embodiments, the primary assembly 14 shown in FIG. 8 is configured to be operated by hand. That is, hand piece 20 is configured to be gripped by hand such that apparatus 10 can be manipulated manually to perform a selected procedure. In some embodiments, the primary assembly 14 shown in FIG. 8 is configured to be operated by a robot. For example, in some embodiments, body 20 is configured for engagement with a robot arm that can be used to manipulate apparatus to perform a selected procedure. In some embodiments, boot 34, shaft 38, base 52, portion 51 and blade 50 can be removed from body 20 by removing boot 34 from body 34. Primary portion 14 can then be used for tissue sterilization as discussed herein after boot 34, shaft 38, base 52, portion 51 and blade 50 are removed from body 20. In some embodiments, a shaft configured for a selected surgical operation can be coupled to body 20 after boot 34, shaft 38, base 52, portion 51 and blade 50 are removed from body 20.

It will be understood that various modifications may be made to the embodiments disclosed herein. Therefore, the above description should not be construed as limiting, but merely as exemplification of the various embodiments. It will be further understood that any feature of one of the embodiments disclosed herein can be incorporated into any of the other embodiments disclosed herein. Those skilled in the art will envision other modifications within the scope and spirit of the claims appended hereto.

What is claimed is:

1. A surgical instrument comprising:
a body extending along a longitudinal axis between opposite proximal and distal end surfaces, the distal end surface comprising a first cavity and a second cavity, the first cavity comprising a first light bulb disposed therein, the second cavity comprising a second light bulb disposed therein;
a shaft comprising opposite proximal and distal ends, the proximal end being coupled to the body; and
a blade coupled to the distal end,
wherein the first light bulb is an ultraviolet (UV) light bulb and the second light bulb is configured to emit visible light.

2. The surgical instrument recited in claim 1, wherein the second light bulb is a light emitting diode.

3. The surgical instrument recited in claim 1, wherein the first cavity comprises a plurality of spaced apart first cavities and the first light bulb comprises a plurality of first light bulbs, the first light bulbs each being disposed in one of the cavities.

4. The surgical instrument recited in claim 1, wherein the first cavities are disposed radially about the longitudinal axis.

5. The surgical instrument recited in claim 1, wherein:
the first cavity comprises a plurality of spaced apart first cavities and the first light bulb comprises a plurality of first light bulbs;
the first light bulbs are each disposed in one of the first cavities;
the second cavity comprises a plurality of spaced apart second cavities and the second light bulb comprises a plurality of second light bulbs; and
the second light bulbs are each disposed in one of the second cavities.

6. The surgical instrument recited in claim 5, wherein the first cavities and the second cavities are disposed radially about the longitudinal axis.

7. The surgical instrument recited in claim 5, wherein the first cavities are each positioned between two of the second cavities and the second cavities are each positioned between two of the first cavities.

8. The surgical instrument recited in claim 1, wherein the cavities each extend parallel to the longitudinal axis such that light emitted by each of the bulbs travels in a direction that is parallel to the longitudinal axis.

9. The surgical instrument recited in claim 1, wherein the cavities each extend at an acute angle relative to the longitudinal axis such that light emitted by each of the bulbs travels in a direction that is at the acute angle relative to the longitudinal axis.

10. The surgical instrument recited in claim 1, wherein the blade is an electrode.

11. The surgical instrument recited in claim 1, wherein the shaft is a first shaft and the instrument further comprises a housing and a second shaft extending from the housing, the second shaft terminating in an electrode, the housing being detachably mountable on the blade and the first shaft, and when the housing is mounted on the blade and the first shaft, the electrode being electrically coupled to the blade.

12. The surgical instrument recited in claim 11, wherein the blade is an electrode.

13. The surgical instrument recited in claim 1, further comprising an insulated portion positioned between the shaft and the blade.

14. A surgical method comprising:
providing a first surgical instrument including a body extending along a longitudinal axis between opposite proximal and distal end surfaces, the distal end surface comprising a first cavity and a second cavity, the first cavity comprising a first light bulb disposed therein, the second cavity comprising a second light bulb disposed therein, the primary assembly comprising a shaft, the shaft comprising opposite proximal and distal ends, the proximal end being coupled to the body, the primary assembly comprising a blade coupled to the distal end, wherein the first light bulb is an ultraviolet (UV) light bulb and the second light bulb is configured to emit visible light;
sterilizing tissue using the first light bulb;
illuminating the sterilized tissue using the second light bulb; and
cutting the sterilized and illuminated tissue with the blade.

15. The method recited in claim 14, further comprising sterilizing tissue adjacent to the cut tissue using the first light bulb.

16. The method recited in claim 14, further comprising providing a second surgical instrument including a housing and a second shaft extending from the housing and terminating in an electrode;
mounting the housing on the blade and the shaft of the first surgical instrument, wherein when so mounted, the electrode is electrically coupled to the blade; and
performing a procedure on tissue with the electrode.

17. The method recited in claim 16, wherein the procedure comprising coagulating tissue.

18. The method recited in claim 16, further comprising removing the housing from the blade such that the second surgical instrument from the first surgical instrument after the procedure is performed.

19. The method recited in claim 18, wherein the procedure comprising coagulating tissue, the method further comprising:
removing the housing from the blade such that the second surgical instrument from the first surgical instrument after the tissue is coagulated and sterilizing the coagulated tissue using the first light bulb.

20. A surgical instrument comprising:
a body extending along a longitudinal axis between opposite proximal and distal end surfaces, the distal end surface comprising a plurality of spaced apart first cavities and a plurality of spaced apart second cavities, the first and second cavities each being disposed radially about the longitudinal axis such that each of the first cavities is positioned between two of the second cavities and each of the second cavities is positioned between two of the first cavities, the first cavities each comprising a first light bulb disposed therein, the second cavities each comprising a second light bulb disposed therein;
a shaft comprising opposite proximal and distal ends, the proximal end being coupled to the body;
an electrode coupled to the distal end; and
an insulating portion positioned between the shaft and the electrode.
wherein the first light bulbs are ultraviolet light bulbs and the second light bulbs are light emitting diodes.
